(19) United States
(12) Patent Application Publication
(10) Pub. No.: US 2015/0045312 A1
(43) Pub. Date: Feb. 12, 2015

(54) Use of Compounds Elevating Glutathione Levels for the Treatment of Autism, Autistic Spectrum Disorders and Fragile X Syndrome

(71) Applicant: Promentis Pharmaceuticals, Inc., Milwaukee, WI (US)

(72) Inventors: Michael Neary, West Allis, WI (US); Chad Beyer, Englewood, CO (US)

(21) Appl. No.: 14/456,834

(22) Filed: Aug. 11, 2014

Related U.S. Application Data
(60) Provisional application No. 61/864,849, filed on Aug. 12, 2013.

(51) Int. Cl. A61K 31/4965; A61K 38/05; A61K 31/497

(52) U.S. Cl. CPC A61K 31/4965; A61K 31/497; A61K 38/05
USPC 514/21.91; 514/252.1; 544/408; 514/255.05; 544/405; 514/252.11; 544/357; 514/255.06; 544/406

(57) ABSTRACT

Methods of treating autism, autistic spectrum disorders or Fragile X syndrome comprised of administering molecules that engage the cystine-glutamate exchange (System x_{c}⁻) and/or elevate the extracellular levels of glutathione in the CNS. Preferred compounds are cysteine/cystine prodrugs or N-acetyl cysteine (NAC) prodrugs.

### FIELD OF THE INVENTION

This invention relates to the use of compounds that engage the cystine-glutamate exchange (System x_{c}⁻) and/or elevate extracellular levels of glutathione in the central nervous system (CNS) of patients with autism, autistic spectrum disorder and/or Fragile X syndrome.

### BACKGROUND OF THE INVENTION

The discovery and development of novel, effective and well-tolerated medicines is an ongoing requirement for the successful treatment and clinical management of patients suffering with autism, autistic spectrum disorders (ASD) and Fragile X syndrome. Recent data suggest that autism and related spectrum disorders are caused, in large part, by alterations in common gene networks and a comparative analysis of gene expression patterns reveal an interrelated set of molecular processes, perturbations in glutamate levels in the brain as well as significant reductions in the levels of glutathione. However, while a tremendous amount of effort has gone into the investigation of these molecular underpinnings of autism and related spectrum disorders, little progress has been made on the development of effective treatment strategies. Thus, there are currently no effective drugs or therapeutic strategies that ameliorate the behavioral or neurochemical disturbances in patients diagnosed with either autism, ASD or Fragile X. Consequently, there is tremendous medical need for the discovery and development of effective and well-tolerated therapeutic interventions for patients suffering with these debilitating and life-altering disorders.

### SUMMARY OF THE INVENTION

The invention provides methods of treating autism, ASD and Fragile X syndrome comprised of administering to a patient in a therapeutically effective amount of a compound that engages the cystine-glutamate exchange (System x_{c}⁻) and/or increases glutathione levels within the brain. In some embodiments, the compound suitable for the purposes of the invention is a cysteine/cystine prodrug or an N-acetyl cysteine (NAC) prodrug.

The compounds that are believed to be effective for the treatment of autism, ASD and Fragile X syndrome, include but are not limited to all of the compounds disclosed and/or claimed in the following patents and patent applications, the disclosures of which are hereby incorporated by reference in their entirety: U.S. Pat. Nos. 7,829,709 and 8,173,809; US Patent Application Publication Numbers 2011/0021533 A1; US 2010/0048587 A1; US 2011/0224156 A1; US 2012/0122793 A1; US 2012/0122792 A1; U.S. patent application Ser. No. 13/465,383 filed May 7, 2012; and U.S. Provisional Patent Application No. 61/512,751, filed Jul. 28 2011.

In a preferred embodiment, the methods of the present invention encompass administering the following compounds:

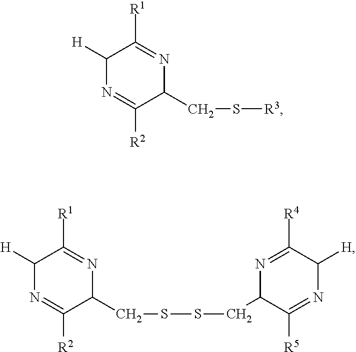

wherein R¹, R², R⁴ and R⁵ are independently selected from OH, ═O, or a branched or straight chain C₁ to C₅ alkoxy group,

with the caveats that when ═O is selected the nitrogen atom adjacent the carbonyl group thusly formed bears a H and a single bond joins the adjacent nitrogen to said carbonyl group and further that the R¹, R², R⁴ and R⁵ that appear in the structure shall be selected to not all be ═O; and

R³ is H, a branched or straight chain C₁ to C₅ alkyl, a nitrobenzenesulfonyl, an aryl thio, an aryl, an alkylthio, an acyl, a benzoyl, a thio acyl, a thio benzoyl, or a benzyl group;

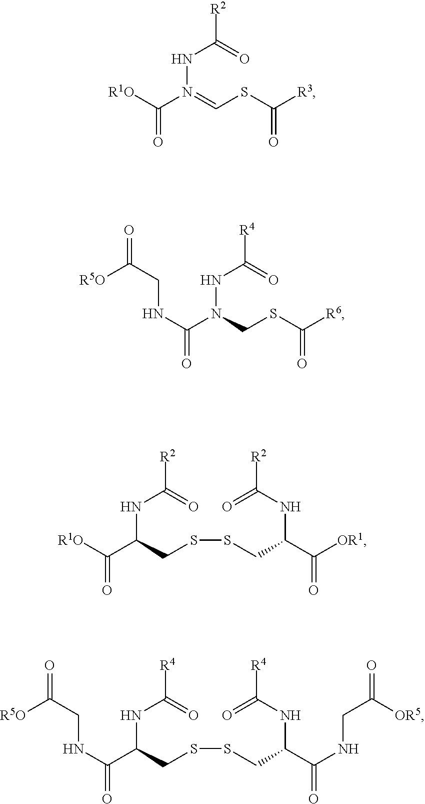

wherein R¹ through R⁶ are independently selected from a branched or straight chain C₁ to C₅ alkyl, a phenyl, or a benzyl group;

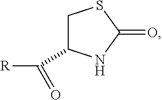

wherein R is selected from the group consisting of:

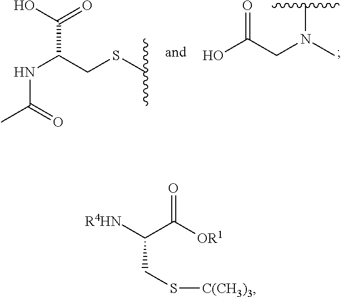

wherein

R¹ is selected from the group consisting of CH₃, CH₂CH₃, CH(CH₃)₂, CH₂-phenyl, and phenyl;

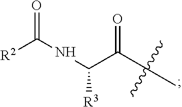

R⁴ is selected from the group consisting of H, C(O)R₂, and

R² is selected from the group consisting of CH₃, CH₂CH₃, CH(CH₃)₂, CH₂-phenyl, and phenyl; and

R³ is selected from the group consisting of H, CH₃, CH₂-phenyl, CH(CH₃)₂, CH₂OH,

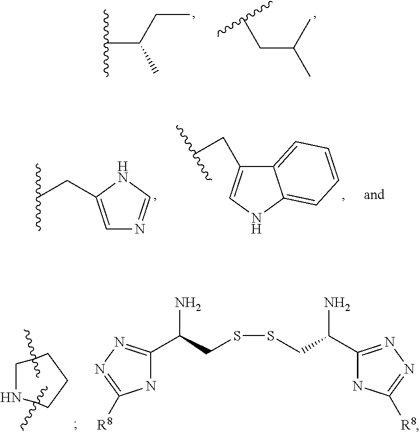

wherein R⁸ is selected from the group consisting of H, CH₃, CH₂CH₃, CH(CH₃)₂ and phenyl.

The invention also encompasses pharmaceutically acceptable salts, esters, bioisosteres, enantiomers, diastereoisomers, mixtures of enantiomers/diastereoisomers, and prodrugs of the provided compounds.

In one embodiment, the methods of the present invention encompass administering the following compound:

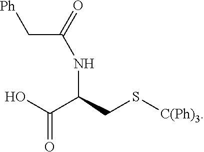

### DETAILED DESCRIPTION OF THE INVENTION

### Definitions

The following definitions are used, unless otherwise described.

The term “prodrugs” refers to compounds, including but not limited to monomers and dimers of the compounds useful for the purposes of the invention, which become under physiological conditions compounds useful for the purposes of the invention or the active moieties of the compounds useful for the purposes of the invention.

The term “active moieties” refers to compounds which are pharmaceutically active in vivo, whether or not such compounds are compounds useful for the purposes of the invention.

The term “composition” means to encompass a product comprising the specified ingredients in the specified amounts, as well as any product which results, directly or indirectly, from a combination of the specified ingredients in the specified amounts.

The term “subject” includes mammals, including humans. The terms “patient” and “subject” are used interchangeably.

In general, unless indicated otherwise, a chemical group referred to anywhere in the specification can be optionally substituted.

The term “therapeutically effective amount” means the amount of a compound that, when administered to a subject for treating autism, ASD or Fragile X syndrome is sufficient to impact such treatment of these disorders. Treating autism and related disorders highlighted in this application does not require the achievement of complete cure. The “therapeutically effective amount” can vary depending on the variety of factors, including the compound, the severity of the disease(s); activity of the specific compound employed; the specific composition employed; the age, body weight, general health, sex and diet of patient; the time of administration, route of administration, and rate of excretion of the specific compound employed; the duration of treatment; drugs used in combination or coincidental with the specific compound employed; and like factors well known in the medical arts. For example, it is well within the skill of the art to start doses of the compound at levels lower than required to achieve the therapeutic effect and to gradually increase the dosage until the desired effect is achieved.

In one embodiment, the terms “treating” or “treatment” refer to ameliorating the symptoms of autism, ASD and Fragile X (i.e., arresting or reducing behavioral disturbances, repetitive behaviors and stereotypes (e.g., hair pulling), cognitive deficits, increasing social interactions or at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease pathology and progression, either physically, (e.g., stabilization of a discernible symptom), physiologically, (e.g., stabilization of a physical parameter), or both. In yet another embodiment, “treating” or “treatment” refers to delaying or preventing the manifestation of the clinical features of the disease(s).

The term “combinational use” as used in the present invention encompasses co-formulations of the two active agents as well as co-administration of two active agents as separate formulations.

### Description of the Invention

The present invention incorporates and is based on new and emerging scientific understanding of autism, autistic spectrum disorders or Fragile X syndrome.

In its broadest embodiment, the invention provides methods of treating these disorders by administering to a patient in need thereof a therapeutically effective amount of a compound that engages the cystine-glutamate exchange (System x_{c}⁻) and/or increases glutathione levels in the brain, brain regions and/or brain cells (neurons). In some embodiments, the compound suitable for the purposes of the invention is a cysteine/cystine prodrug or an N-acetyl cysteine (NAC) prodrug.

The compounds that are believed to be effective for the treatment of autism and related spectrum disorders, include but are not limited to all of the compounds disclosed and/or claimed in the following patents and patent applications, the disclosures of which are hereby incorporated by reference in their entirety: U.S. Pat. Nos. 7,829,709 and 8,173,809; US Patent Application Publication Numbers 2011/0021533 A1; US 2010/0048587 A1; US 2011/0224156 A1; US 2012/0122793 A1; US 2012/0122792 A1; U.S. patent application Ser. No. 13/465,383 filed May 7 2012; and U.S. Provisional Patent Application No. 61/512,751, filed Jul. 28 2011.

On the biochemical level, the regulation of oxidative stress and glutathione levels has emerged as an important clinical feature of autism where alterations in oxidative stress have been implicated as a possible link to disease severity (Kessler, R. C., et al. (2005) Arch Gen Psychiatry 62, 617-627; Gilman, S. R. et al. (2012) Nat Neurosci 15, 1723-1738; Dean, O. M. et al. (2009) Curr Med Chem 16, 2965-2976. Similarly, improper redox balance is suggested to mediate the severity of autism (Kern, J. K. & Jones, A. M. (2006) J Toxicol Environ Health B Crit Rev 9, 485-499; James, S. J. et al. (2006) Am J Med Genet B Neuropsychiatr Genet 141B, 947-956; James, S. J. et al. (2009) FASEB J 23, 2374-2383. Specifically, mutations in genes regulating many different steps in the production of glutathione, the body's primary mediator of oxidative stress and subsequent mitochondrial dysfunction, have been identified in individuals with autism (James, S. J. et al. (2006) Am J Med Genet B Neuropsychiatr Genet 141B, 947-956; James, S. J. et al. (2009) FASEB J 23, 2374-2383; Yorbik, O. et al. (2002) Prostaglandins Leukot Essent Fatty Acids 67, 341-343; Deth, R et. al. (2008) Neuro Toxicology 29, 190-201). Furthermore, alterations in glutamatergic signaling also underlie, in part, the clinical symptoms of autism, ASD and Fragile X syndrome. Thus, alterations in glutamate signaling have been demonstrated in autism, where glutatmate levels are elevated in both the cerebrospinal fluid and serum (Tirouvanziam, R. et al. (2012) J Autism Dev Disord 42, 827-836; Shinohe, A. et al. (2006) Prog Neuropsychopharmacol Biol Psychiatry 30, 1472-147; Moreno-Fuenmayor, H. et al. (1996) Invest Clin 37, 113-128; Aldred, S. et al. (2003) J Autism Dev Disord 33, 93-99). Moreover, genetic studies have demonstrated a link between autism and glutamate receptor genes, and genes associated with glutamatergic transmission (Purcell, A. E. et al. (2001) Neurology 57, 1618-1628). In sum, this data offers compelling evidence that autism and related spectrum disorders may be sensitive to similar therapeutic treatments that engage the cystine-glutamate exchange (System x_{c}⁻) and/or elevate extracellular levels of glutathione in the central nervous system (CNS) of patients. One approach to regulate glutathione in the brain is through the key antiporter system Xc−. System Xc− regulates the free uptake of N-acetyl cysteine (NAC), a key precursor in the production of cellular glutathione, in exchange for the release of glutamate. Thus, NAC and system Xc− mediate both oxidative stress and glutamatergic signaling. Not surprisingly, administration of NAC has been shown to modestly improve symptoms of many mental disorders including schizophrenia, bipolar disorder, obsessive-compulsive disorder, and most recently autism (Berk, M. (2013) Trends Pharmacol Sci 34, 167-177; Hardan, A Y et al. (2012) Biol Psychiatry 71, 956-961).

Additionally, in a recent clinical trial, NAC was shown in autistic patients when added to risperidone treatment to significantly decrease irritability versus risperidone+placebo (Ghanizadeh, A et al. (2013) BMC Psychiatry 13, 196).

Based on these studies, the inventors of the present application reasonably believe that increasing System x_{c}− activity and increasing glutathione levels may be clinically effective for treating autism, ASD and Fragile X syndrome in a manner that may target an important underlying pathophysiological process. Further, pharmacological strategies designed to ultimately reduce or prevent the formation of ROS, may have profound disease modifying potential for patients with autism.

The compounds disclosed and/or claimed in U.S. Pat. Nos. 7,829,709 and 8,173,809; US Patent Application Publication Numbers 2011/0021533 A1; US 2010/0048587 A1; US 2011/0224156 A1; US 2012/0122793 A1; US 2012/0122792 A1; U.S. patent application Ser. No. 13/465,383 filed May 7 2012; and U.S. Provisional Patent Application No. 61/512,751, filed Jul. 28 2011 are either cysteine/cystine prodrugs and/or NAC prodrugs and/or the compounds that increase glutathione levels. Accordingly, this invention provides methods of using these compounds for the treatment of autism and related disorders comprising administering to a patient in need thereof a therapeutically effective amount of one or more of these compounds.

Specifically, these compounds are believed to be superior to glutathione and NAC for the treatment of autism, ASD and Fragile X syndrome because glutathione and NAC cannot be used effectively in the clinic because they do not readily enter the CNS and possess poor solubility characteristics.

In a preferred embodiment, the methods of the present invention encompass administering the following compound:

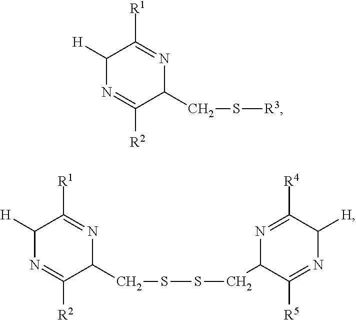

wherein R¹, R², R⁴ and R⁵ are independently selected from OH, ═O, or a branched or straight chain C₁ to C₅ alkoxy group, with the caveats that when ═O is selected the nitrogen atom adjacent the carbonyl group thusly formed bears a H and a single bond joins the adjacent nitrogen to said carbonyl group and further that the R¹, R², R⁴ and R⁵ that appear in the structure shall be selected to not all be ═O; and

R³ is H, a branched or straight chain C₁ to C₅ alkyl, a nitrobenzenesulfonyl, an aryl thio, an aryl, an alkylthio, an acyl, a benzoyl, a thio acyl, a thio benzoyl, or a benzyl group;

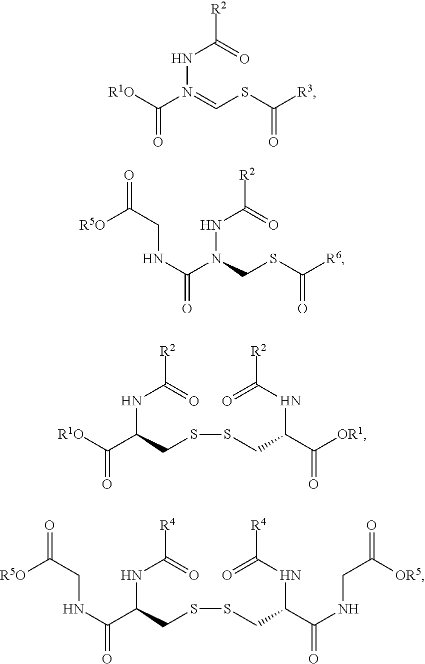

wherein R¹ through R⁶ are independently selected from a branched or straight chain C₁ to C₅ alkyl, a phenyl, or a benzyl group;

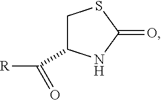

wherein R is selected from the group consisting of:

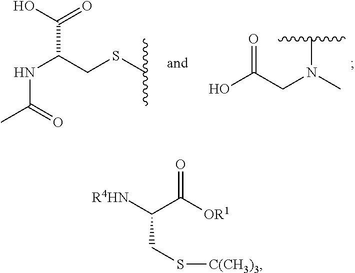

wherein

R¹ is selected from the group consisting of CH₃, CH₂CH₃, CH(CH₃)₂, CH₂-phenyl, and phenyl;

R⁴ is selected from the group consisting of H, C(O)R₂, and

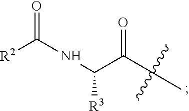

R² is selected from the group consisting of CH₃, CH₂CH₃, CH(CH₃)₂, CH₂-phenyl, and phenyl; and

R³ is selected from the group consisting of H, CH₃, CH₂-phenyl, CH(CH₃)₂, CH₂OH,

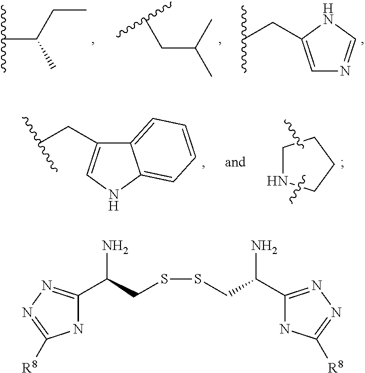

wherein R⁸ is selected from the group consisting of H, CH₃, CH₂CH₃, CH(CH₃)₂ and phenyl.

The invention also encompasses pharmaceutically acceptable salts, esters, bioisosteres, enantiomers, diastereoisomers, mixtures of enantiomers/diastereoisomers, and prodrugs of the provided compounds.

In one embodiment, the methods of the present invention encompass administering the following compound:

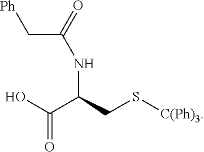

On the basis of specific parameters, this compound is believed to be best suited to cross into the CNS.

Using an in vitro screening assay conducted in human glial cells from brain astrocytoma (1321N1), a cell line with high system x_{c}− expression, the compound with the following formula:

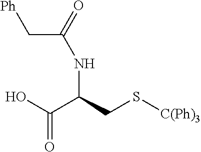

was found to drive system x_{c}− as evidenced by a robust and significant decrease in the uptake of ¹⁴C-cystine and significant elevations in ³H-glutamate.

On the basis of specific parameters, these compounds are believed to be best suited to cross into the CNS.

Using an in vitro screening assay conducted in human glial cells from brain astrocytoma (1321N1), a cell line with high System x_{c}− expression, the compound with the following formula:

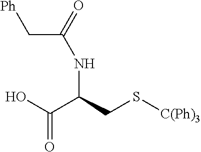

was found to drive System x_{c}− as evidenced by a robust and significant decrease in the uptake of ¹⁴C-cystine and significant elevations in ³H-glutamate.

In efficacy models of neuropsychiatric disease activity (pre-pulse inhibition (PPI) and elevated plus maze (EPM)), this compound is orally-active and produces significant behavioral effects in both of these models. Further, in the PPI model, this compound elicits an antipsychotic-like response (at a comparable dose range) similar to the current standard of care in human patients (i.e., clozapine).

On the basis of these data, and additional in vitro selectivity experiments at nearly 50 separate enzymes, transporters, and GPCRs (data not shown), the inventors believe that the most preferred compounds exemplified by the structural formulas above increase System x_{c}− activity and increase glutathione levels, and therefore are suitable for the treatment of autism, ASD and Fragile X.

The present invention also provides pharmaceutical compositions that comprise the compounds suitable for the purposes of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally, intracisternally, intravaginally, transdermally (e.g. using a patch), transmucosally, sublingually, pulmonary, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

In another aspect, the present invention provides a pharmaceutical composition comprising a compound suitable for the purposes of the present invention and a physiologically tolerable diluent. The present invention includes one or more compounds as described above formulated into compositions together with one or more non-toxic physiologically tolerable or acceptable diluents, carriers, adjuvants or vehicles that are collectively referred to herein as diluents, for parenteral injection, for intranasal delivery, for oral administration in solid or liquid form, for rectal or topical administration, among others.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds suitable for the purposes of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound suitable for the purposes of this invention include powders, sprays, ointments and inhalants. The active compound is mixed under sterile conditions with a pharmaceutically acceptable carrier and any needed preservatives, buffers or propellants which can be required. Ophthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the autism or related disorder and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds suitable for the purposes of the present invention can be employed in pure form or, where such forms exist, in pharmaceutically acceptable salt, ester or prodrug form. Alternatively, the compound can be administered as a pharmaceutical composition containing the compound of interest in combination with one or more pharmaceutically acceptable excipients.

The total daily dose of the compounds suitable for the purposes of this invention administered to a human or lower animal may range from about 0.0001 to about 1000 mg/kg/day. If desired, the effective daily dose can be divided into multiple doses for purposes of administration; consequently, single dose compositions may contain such amounts or submultiples thereof to make up the daily dose.

The methods of the invention can be used in combination with the use of other drugs known for the treatment of autism, ASD and Fragile X, including but not limited to, various antipsychotic treatment regimens (clozapine or Haldol).

1. A method of treating autism, autistic spectrum disorders or Fragile X syndrome comprising administering to a patient in need thereof a therapeutically effective amount of an N-acetyl cysteine (NAC) prodrug.

2. (canceled)

3. A pharmaceutical composition for the treatment of autism, autistic spectrum disorders or Fragile X syndrome comprising the NAC prodrug of claim 1.

4. (canceled)

5. A method of treating autism, autistic spectrum disorders or Fragile X syndrome comprising administering to a patient in need thereof a therapeutically effective amount of a compound selected from 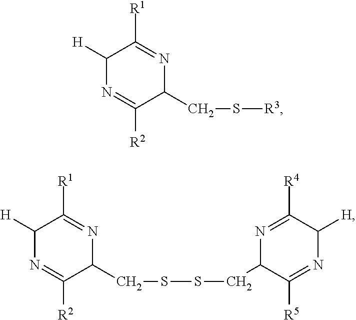
wherein R¹, R², R⁴ and R⁵ are each independently selected from OH, ═O, or a branched or straight chain C₁ to C₅ alkoxy group,
with the caveats that when ═O is selected the nitrogen atom adjacent the carbonyl group thusly formed bears a H and a single bond joins the adjacent nitrogen to said carbonyl group and further that the R¹, R², R⁴ and R⁵ that appear in the structure shall be selected to not all be ═O and
R³ is H, a branched or straight chain C₁ to C₅ alkyl, a nitrobenzenesulfonyl, an aryl thio, an aryl, an alkylthio, an acyl, a benzoyl, a thio acyl, a thio benzoyl, or a benzyl group; 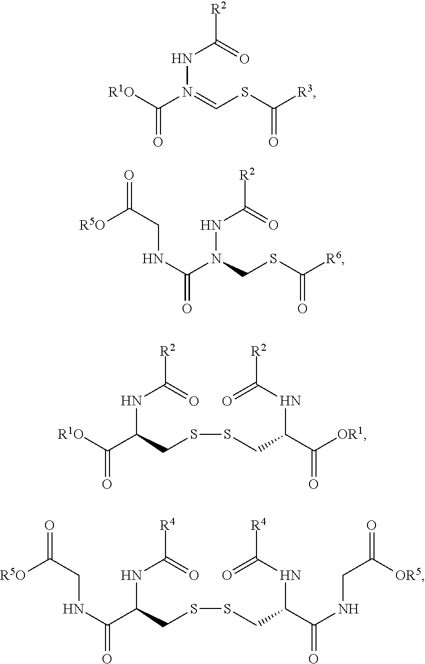
wherein R¹ through R⁶ are each independently selected from a branched or straight chain C₁ to C₅ alkyl, a phenyl, or a benzyl group; 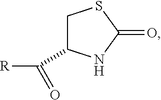
wherein R is selected from: 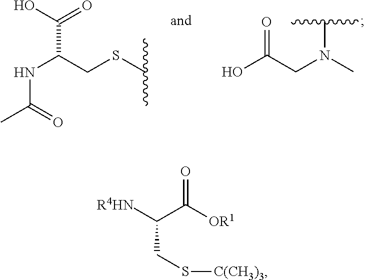
wherein
R¹ is selected from CH₃, CH₂CH₃, CH(CH₃)₂, CH₂-phenyl, and phenyl;
R⁴ is selected from H, C(O)R₂, and 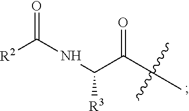
R² is selected from CH₃, CH₂CH₃, CH(CH₃)₂, CH₂-phenyl, and phenyl; and
R³ is selected from H, CH₃, CH₂-phenyl, CH(CH₃)₂, CH₂OH, 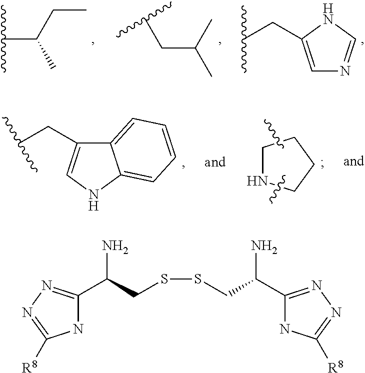
wherein R⁸ is selected from H, CH₃, CH₂CH₃, CH(CH₃)₂ and phenyl,
or pharmaceutically acceptable salts, esters, bioisosteres, enantiomers, diastereoisomers, mixtures of enantiomers/diastereoisomers, or prodrugs thereof.

6. A pharmaceutical composition for the treatment of autism, autistic spectrum disorders or Fragile X syndrome comprising the compound of claim 5.
